(19) United States
(12) Patent Application Publication
(10) Pub. No.: US 2016/0325499 A1
(43) Pub. Date: Nov. 10, 2016

(54) SYSTEMS AND METHODS FOR PRODUCING AND APPLYING TISSUE-RELATED STRUCTURES

(71) Applicant: David Muller, Boston, MA (US)

(72) Inventor: David Muller, Boston, MA (US)

(21) Appl. No.: 15/109,731

(22) PCT Filed: Jan. 5, 2015
(86) PCT No.: PCT/US2015/010121

§ 371 Date: Jul. 5, 2016

Related U.S. Application Data
(60) Provisional application No. 61/923,734, filed on Jan. 5, 2014.

(51) Int. Cl. B29C 67/00; A61L 27/38; A61F 2/14; A61L 27/36

(52) U.S. Cl. CPC B29C 67/0085; A61L 27/3641; A61L 27/3804; A61F 2/142; B29C 67/0059; B29C 67/0088; A61L 2430/16; A61F 2240/002; B29K 2071/02

(57) ABSTRACT

Embodiments produce and apply tissue-related structures in connection with various medical treatments. Such structures can be applied, as grafts, implants, scaffolds, etc., to replace, modify, or engineer tissue in the body. For example, such structures can be employed to reshape the cornea in order to correct vision. One example includes a tissue cell source including tissue cells in a fluid and a printer configured to deposit the tissue cells in a three-dimensional arrangement to form a tissue cell-based structure. Another example includes a source including a photoreactive liquid precursor, an application system configured to deposit the photoreactive liquid precursor in one or more applications to form a three-dimensional polymer-based structure, and an illumination system configured to deliver light to the photoreactive liquid precursor deposited by the application system and to solidify the photoreactive liquid precursor into the three-dimensional polymer-based structure.

### CROSS-REFERENCE TO RELATED APPLICATIONS

This application claims priority to U.S. Provisional Patent Application No. 61/923,734, filed Jan. 5, 2014, the contents of which are incorporated entirely herein by reference.

### BACKGROUND

1. Field

The disclosed subject matter pertains generally to medical treatments, and more particularly, to systems and methods for producing and applying tissue-related structures in connection with various medical treatments, for example, implantable structures for treating corneal disorders.

2. Description of Related Art

A variety of eye disorders, such as myopia, keratoconus, and hyperopia, involve abnormal shaping of the cornea. Many procedures correct such disorders by changing structural aspects of the cornea. For example, laser-assisted in-situ keratomileusis (LASIK) reshapes the cornea surgically so that light traveling through the cornea is properly focused onto the retina located in the back of the eye.

### SUMMARY

According to aspects of the present disclosure, systems and methods produce and apply tissue-related structures in connection with various medical treatments. Such structures can be applied, as grafts, implants, scaffolds, etc., to replace, modify, or engineer tissue in the body. For example, such structures can be employed to reshape the cornea in order to correct vision.

According to an example embodiment, a system for producing a tissue-related structure includes a tissue cell source including tissue cells in a fluid. The system also includes a printer coupled to the tissue cell source and configured to deposit the tissue cells in a three-dimensional arrangement to form a tissue cell-based structure. The tissue cell fluid has characteristics that allow the tissue cells to be deposited via the printer. In addition, the system includes a computing system coupled to the printer and configured to control the printer to deposit the tissue cells at selected positions defined by the arrangement.

According to another example embodiment, a system for producing a tissue-related structure includes a source including a photoreactive liquid precursor. The system also includes an application system coupled to the source and configured to deposit the photoreactive liquid precursor in one or more applications to form a three-dimensional polymer-based structure. The photoreactive liquid precursor has characteristics that allow the photoreactive liquid precursor to be deposited via the application system. In addition, the system includes an illumination system configured to deliver light to the photoreactive liquid precursor deposited by the application system and to solidify the photoreactive liquid precursor into the three-dimensional polymer-based structure.

According to a yet another example embodiment, a method for producing a tissue-related structure includes determining a three-dimensional arrangement of tissue cells to form a tissue cell-based structure. The method also includes coupling a printer to a tissue cell source including tissue cells in a fluid. In addition, the method includes depositing, with a printer, the tissue cells according to the arrangement to form the tissue cell-based structure. The tissue cell fluid has characteristics that allow the tissue cells to be deposited via the printer.

According to a further embodiment, a method for producing a tissue-related structure includes determining one or more applications of a photoreactive liquid precursor to form a three-dimensional polymer-based structure. The method also includes coupling an application system to a source including a photoreactive liquid precursor. In addition, the method includes depositing, with the application system, the photoreactive liquid precursor according to the one or more determined applications to form the three-dimensional polymer-based structure. The photoreactive liquid precursor has characteristics that allow the photoreactive liquid precursor to be deposited via the application system. Moreover, the method includes delivering light, with an illumination system, to the photoreactive liquid precursor deposited by the application system and to solidify the photoreactive liquid precursor into the three-dimensional polymer-based structure.

Additional aspects of the present disclosure will be apparent to those of ordinary skill in the art in view of the detailed description of various embodiments, which is made with reference to the drawings, a brief description of which is provided below.

### BRIEF DESCRIPTION OF THE DRAWINGS

FIG. 1 illustrates an example of a three-dimensional (3D) printing system that produces highly defined cell-based structures, according to aspects of the present disclosure.

FIG. 2 illustrates an example of a 3D printing system that produce highly defined cell-based structures of a corneal collagen matrix with keratocytes, according to aspects of the present disclosure.

FIG. 3 illustrates an example of a 3D printing system that employs two-photon polymerization to produce highly defined polymer-based structures for medical applications, according to aspects of the present disclosure.

FIG. 4 illustrates an example of a 3D printing system that employs two-photon (or multi-photon) polymerization to produce highly defined polymer-based structures in vivo for medical applications, according to aspects of the present disclosure.

While example embodiments are susceptible to various modifications and alternative forms, a specific embodiment thereof has been shown by way of example in the drawings and will herein be described in detail. It should be understood, however, that it is not intended to limit the example embodiments to the particular forms disclosed, but on the contrary, the intention is to cover all modifications, equivalents, and alternatives falling within the spirit of the present disclosure.

### DETAILED DESCRIPTION

According to aspects of the present disclosure, systems and methods produce and apply tissue-related structures in connection with various medical treatments. Such structures can be applied, as grafts, implants, scaffolds, etc., to replace, modify, or engineer tissue in the body.

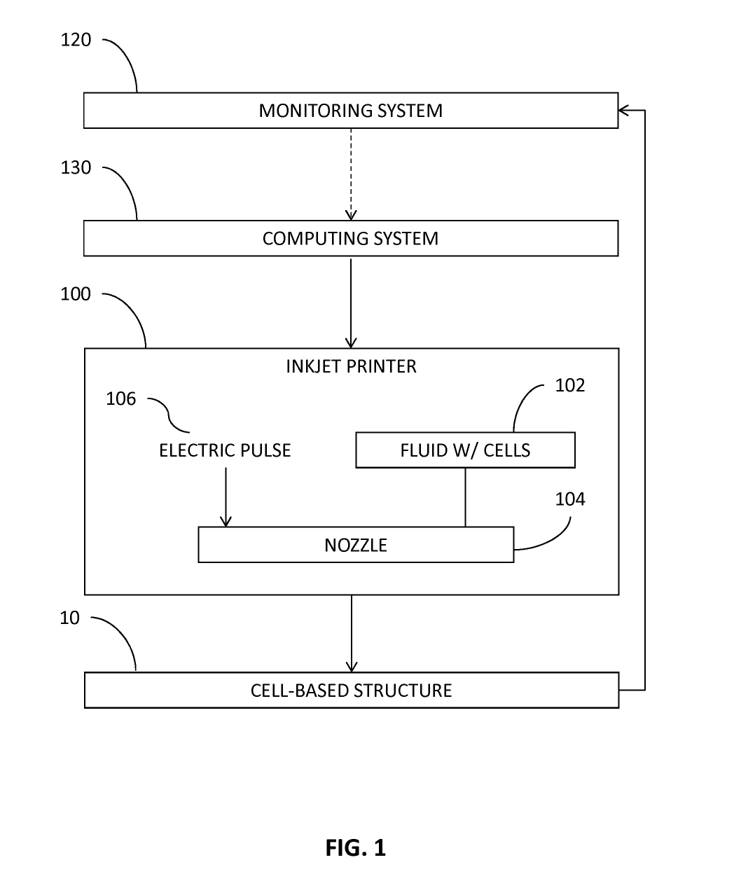

In some embodiments, aspects of three-dimensional (3D) printing are employed to produce highly defined cell-based structures using cells taken from various types of tissue. It has been shown that an inkjet printer can be used to print cells taken from body tissue. The printed cells can remain healthy and survive and grow in culture. As shown schematically in FIG. 1, to produce a cell-based structure 10, aspects of the present disclosure may employ an inkjet printer device 100, e.g., piezoelectric inkjet printer, which ejects cells in a fluid 102 through a sub-millimeter diameter nozzle 104 in response to a specific electrical signal 106, e.g., pulse. The fluid 102 with the cells is produced with the appropriate characteristics, e.g., viscosity and surface tension, to be ejected effectively from the nozzle 104. The inkjet printer device 100 is controlled to deposit the cells in a specified 3D arrangement that forms the cell-based structure 10. A monitoring system 120, including high speed video technology for instance, may be employed to obtain high resolution images of the printing process and to optimize the printing process. In addition, a computing system 130 may control the operation of the inkjet printer device 100 to deposit the cells according to the specified arrangement. For example, the computing system 130 may trigger the electrical signal 106 to cause the nozzle 104 of a piezoelectric inkjet printer to deposit the cells at selected (x, y, z) positions. The selected (x, y, z) positions can be programmed into instructions stored on computer-readable media for the computing system 130. The computing system 130 may optionally employ information from the monitoring system 120 as feedback to control the inkjet printer device 100 during the printing process.

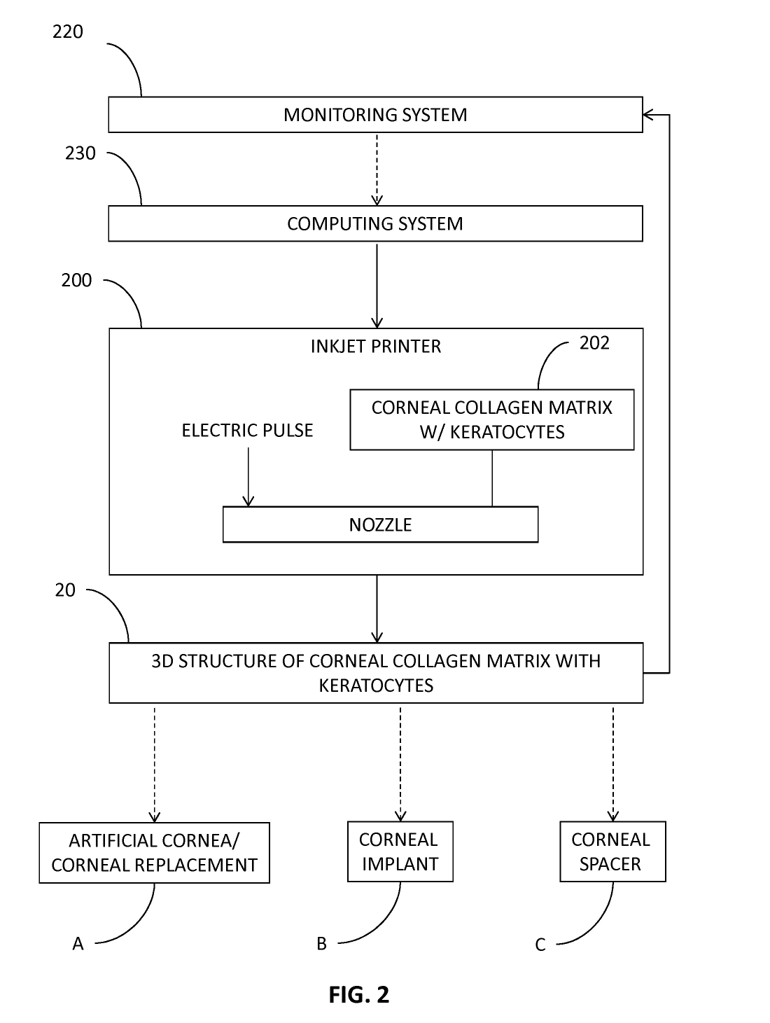

In an example embodiment, aspects of 3D printing are employed to produce structures using corneal cells. These structures can then be employed to treat disorders relating to the cornea. As shown in FIG. 2, an inkjet printer device 200 prints a 3D cell-based structure 20 from a fluid source 202 containing a corneal collagen matrix with keratocytes. The inkjet printer device 200 deposits and organizes the cells into an arrangement (corneal cellular matrix) that gives the corneal 3D structure 20 the necessary characteristics to be used in vivo. For example, the corneal 3D structure 20 may be configured for use as (A) an artificial cornea/cornea replacement; (B) a corneal implant (onlay and inlay) to reshape the cornea for refractive correction; or (C) a spacer for other corneal restructuring. Aspects of corneal implant systems and methods are described, for example, in U.S. patent application Ser. No. 14/152,425, filed on Jan. 10, 2014, the contents of which are incorporated entirely herein by reference. As FIG. 2 also illustrates, a monitoring system 220, including high speed video technology for instance, may be employed to obtain high resolution images of the printing process and to optimize the printing process. In addition, a computing system 230 may control the operation of the inkjet printer device 200 to deposit the cells according to the specified arrangement. The arrangement for the deposited cells can be programmed into instructions stored on computer-readable media for the computing system 230. The computing system 230 may optionally employ information from the monitoring system 220 as feedback to control the inkjet printer device 200 during the printing process.

In other embodiments, aspects of 3D printing are employed to produce highly defined polymer-based structures, which can be used, for example, as scaffolds for tissue engineering. In particular, aspects of the present disclosure can employ two-photon polymerization to make small-scale solid structures from a photoreactive liquid precursor. An inkjet printer may be employed in an application system to apply the photoreactive liquid precursor to define the structures. The liquid precursor contains chemicals that react to light, turning the liquid into a solid polymer. 3D structures are formed by exposing the liquid precursors to targeted amounts of light.

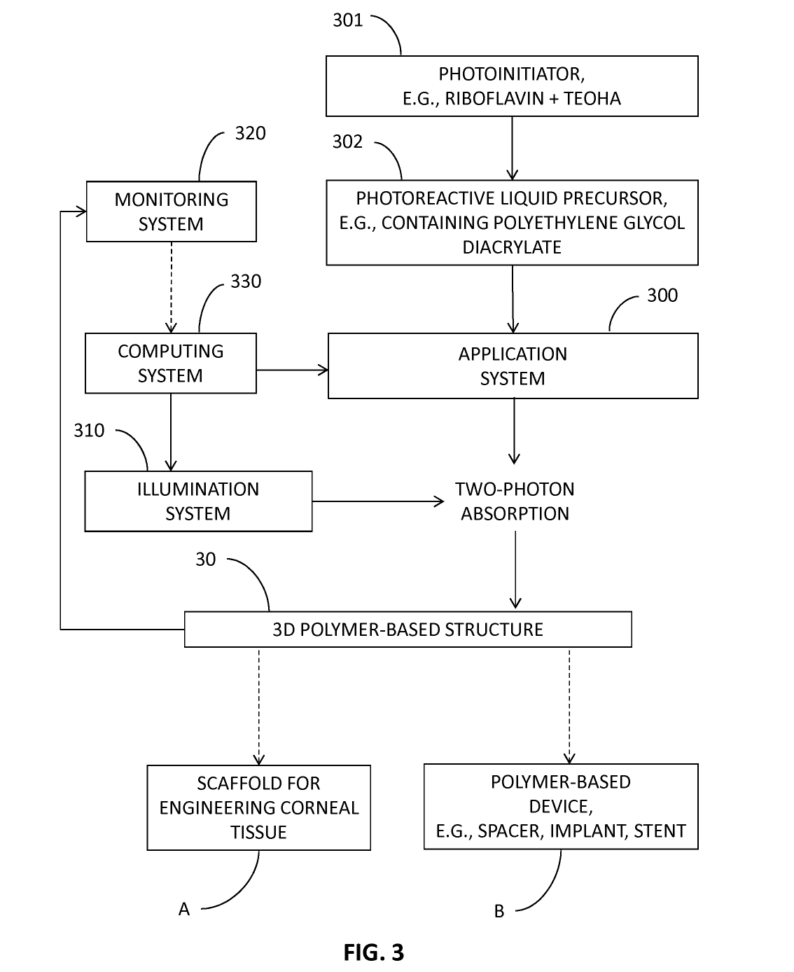

In some embodiments, biocompatible photoinitiators, such as riboflavin, are mixed with the precursor materials to make the liquid precursor photoreactive. In an example shown in FIG. 3, riboflavin is combined with triethanolamine (TEOHA) to provide a biocompatible photoinitiator 301 for two-photon polymerization processing of a photoreactive precursor 302, e.g., containing polyethylene glycol diacrylate. The riboflavin-TEOHA mixture causes the polyethylene glycol diacrylate to cross-link when it receives the energy from two simultaneous photons of identical or different wavelengths from an illumination system 310 (two photon absorption). In some cases, the illumination system 310 produces ultraviolet (UV) light for two-photon polymerization. As such, a solid 3D polymer-based 30 structure is formed. For example, using the riboflavin-TEOHA mixture as a photoinitiator for two-photon polymerization produces effective scaffolds for the seeding of cells for tissue engineering. Of course, this process can also be used to form other structures for medical treatments, e.g., micro-needles or other implantable drug-delivery devices, etc. In addition, other photoinitiators, such as Irgacure® 369 or Irgacure® 2959 may be employed to initiation cross-linking for the polymerization.

As FIG. 3 also shows, an application system 300 applies the photoreactive liquid precursor 302 for exposure to the light from the illumination system 310. A series of applications of the photoreactive liquid precursor 302 and corresponding exposures to light can be employed to form the 3D polymer-based structure. A monitoring system 320, including high speed video technology for instance, may be employed to obtain high resolution images of, and to optimize, the application and polymerization process. In addition, a computing system 330 may control the operation of the application system 300 and the illumination system 310. The arrangement for the 3D polymer-based structure can be programmed into instructions stored on computer-readable media for the computing system 330. The computing system 330 may optionally employ information from the monitoring system 320 as feedback to control the application system 300 and the illumination system 310 during the application and polymerization process.

In example applications of the system shown in FIG. 3, aspects of 3D printing with two-photon polymerization are employed to produce structures to treat disorders relating to the eye. Such structures may be used as scaffolds for seeding corneal cells and engineering corneal tissue for replacement cornea or corneal implants for refractive correction (A). Alternatively, such structures may be used as polymer spacers for restructuring aspects of the cornea, polymer corneal implants (onlay or inlay) for making refractive corrections, or polymer stents in Schlemm's canal to relieve intraocular pressure for the treatment of glaucoma (B).

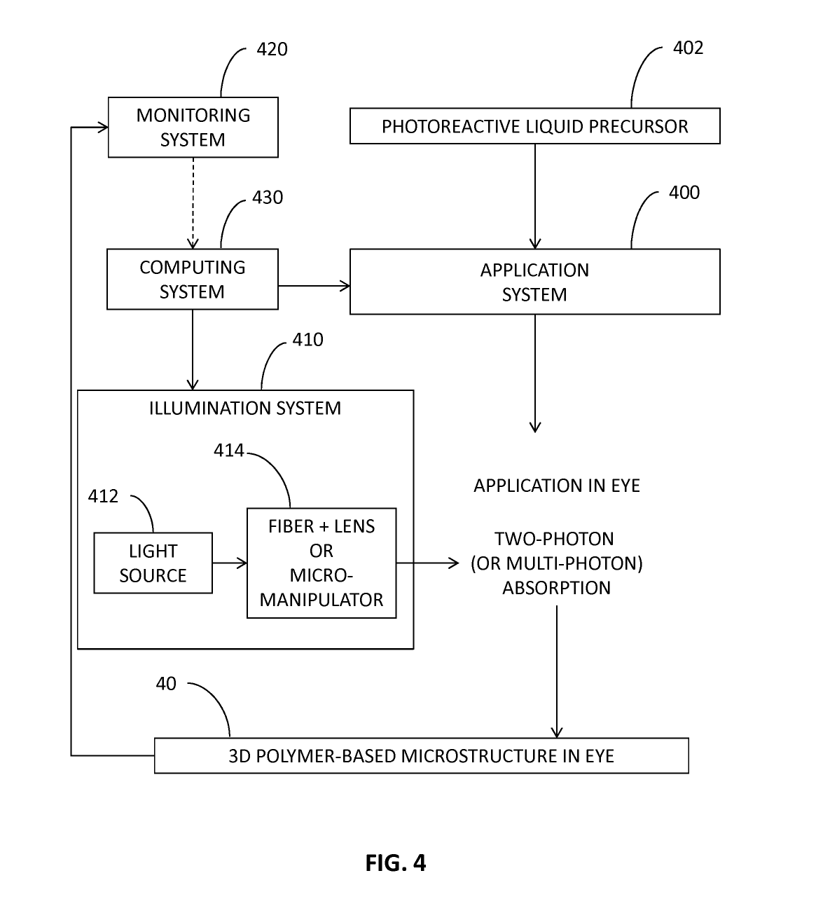

In some embodiments, microstructures may be formed in vivo with two-photon polymerization. For example, as shown schematically in FIG. 4, a microstructure 40 may be formed in the eye by applying a photoreactive liquid precursor 402 with an application system 400 (e.g., syringe) and applying light from a light source 412 of an appropriate (non-damaging) wavelength in an illumination system 410. Effective polymerization occurs with two-photon (or even multi-photon, e.g., three-photon) absorption of the selected wavelength. This in vivo process may involve exposing a surface, e.g., of the brain, artery, etc., which can then be modified accordingly. The light may be delivered through a specially configured delivery device 414 of the illumination system 410. The delivery device 414 may be an optical fiber with an appropriate focusing lens at the distal end to allow for two-photon absorption. Alternatively, the delivery device 414 may be a micro-manipulator that can deliver the light according to the desired pattern to create the 3D structures by polymerization. Some embodiments may employ Digital Micromirror Device (DMD) technology to modulate the application of the light spatially as well as a temporally. Using DMD technology, a controlled light source projects the initiating light in a precise spatial pattern that is created by microscopically small mirrors laid out in a matrix on a semiconductor chip, known as a (DMD). Each mirror represents one or more pixels in the pattern of projected light.

A monitoring system 420, including high speed video technology for instance, may be employed to obtain high resolution images of, and to optimize, the application and polymerization process. In addition, a computing system 430 may control the operation of the application system 400 and the illumination system 410. The arrangement for the 3D polymer-based structure can be programmed into instructions stored on computer-readable media for the computing system 430. The computing system 430 may optionally employ information from the monitoring system 420 as feedback to control the application system 400 and the illumination system 410 during the application and polymerization process.

As described above, example embodiments may employ aspects of multi-photon (two-photon, three-photon, etc.) absorption. In particular, rather than delivering a single photon of a particular wavelength to the photoreactive liquid precursor, the illumination system delivers multiple photons of longer wavelengths, i.e., lower energy, that combine to initiate a photoreaction. Advantageously, longer wavelengths are scattered to a lesser degree than shorter wavelengths, which allows longer wavelengths of light to penetrate a substrate more efficiently than shorter wavelength light. For example, in some embodiments using riboflavin as a photoinitiator, two photons may be employed, where each photon carries approximately half the energy necessary to cause cross-linking activity. When a molecule simultaneously absorbs both photons, it absorbs enough energy to generate the cross-linking activity. Embodiments may also utilize lower energy photons such that a molecule must simultaneously absorb, for example, three, four, or five, photons to initiate a photoreaction. The probability of the near-simultaneous absorption of multiple photons is low, so a high flux of photons may be required, and the high flux may be delivered through a femtosecond laser for instance. Because multiple photons are absorbed for photoreaction by the molecule, the probability for photoreaction increases with intensity. Therefore, greater photoreaction results where the delivery of light is tightly focused compared to where it is more diffuse. The illumination system may deliver a laser beam to the photoreactive liquid precursor. Effectively, photoreaction is restricted to the smaller focal volume where the light is delivered with a high flux. This localization advantageously allows for more precise control over the location of polymerization.

Embodiments employing multi-photon absorption can also optionally employ multiple beams of light simultaneously. For example, a first and a second beam of light can each be directed from the illumination system to an overlapping region the application of the photoreactive liquid precursor. The region of intersection of the two beams of light can be a volume where polymerization is desired to occur. Multiple beams of light can be delivered using aspects of the illumination system to split a beam of light emitted from the light source and direct the resulting multiple beams of light to the overlapping region. In addition, embodiments employing multi-photon absorption can employ multiple light sources, each emitting a beam of light, such that the multiple resulting beams of light overlap or intersect in a volume where polymerization is desired to occur. Aspects of the present disclosure employing overlapping beams of light to achieve multi-photon microscopy may provide an additional approach to controlling the polymerization of the according to a desired three-dimensional structure.

The embodiments described herein may employ various computing systems for processing information and controlling aspects of various devices. The processor(s) of a computing system may be implemented as a combination of hardware and software elements. The hardware elements may include combinations of operatively coupled hardware components, including microprocessors, communication/networking interfaces, memory, signal filters, circuitry, etc. The processors may be configured to perform operations specified by the software elements, e.g., computer-executable code stored on computer readable medium. The processors may be implemented in any device, system, or subsystem to provide functionality and operation according to the present disclosure. The processors may be implemented in any number of physical devices/machines. Indeed, parts of the processing of the example embodiments can be distributed over any combination of processors for better performance, reliability, cost, etc.

The physical devices/machines can be implemented by the preparation of integrated circuits or by interconnecting an appropriate network of conventional component circuits, as is appreciated by those skilled in the electrical art(s). The physical devices/machines, for example, may include field programmable gate arrays (FPGA's), application-specific integrated circuits (ASIC's), digital signal processors (DSP's), etc. The physical devices/machines may reside on a wired or wireless network, e.g., LAN, WAN, Internet, cloud, near-field communications, etc., to communicate with each other and/or other systems, e.g., Internet/web resources.

Appropriate software can be readily prepared by programmers of ordinary skill based on the teachings of the example embodiments, as is appreciated by those skilled in the software arts. Thus, the example embodiments are not limited to any specific combination of hardware circuitry and/or software. Stored on one computer readable medium or a combination of computer readable media, the computing systems may include software for controlling the devices and subsystems of the example embodiments, for driving the devices and subsystems of the example embodiments, for enabling the devices and subsystems of the example embodiments to interact with a human user (user interfaces, displays, controls), etc. Such software can include, but is not limited to, device drivers, operating systems, development tools, applications software, etc. A computer readable medium further can include the computer program product(s) for performing all or a portion of the processing performed by the example embodiments. Computer program products employed by the example embodiments can include any suitable interpretable or executable code mechanism, including but not limited to complete executable programs, interpretable programs, scripts, dynamic link libraries (DLLs), applets, etc. The processors may include, or be otherwise combined with, computer-readable media. Some forms of computer-readable media may include, for example, a hard disk, any other suitable magnetic medium, CD-ROM, CDRW, DVD, any other suitable optical medium, RAM, PROM, EPROM, FLASH-EPROM, any other suitable memory chip or cartridge, a carrier wave, or any other suitable medium from which a computer can read.

It should be understood that arrangements described herein are for purposes of example only. As such, those skilled in the art will appreciate that other arrangements and other elements (e.g., machines, interfaces, functions, orders, groupings of functions, etc.) can be used instead, and some elements may be omitted altogether according to the desired results. Further, many of the elements that are described are functional entities that may be implemented as discrete or distributed components or in conjunction with other components, in any suitable combination and location.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

1. A system for producing a tissue-related structure, comprising:
a tissue cell source including tissue cells in a fluid;
a printer coupled to the tissue cell source and configured to deposit the tissue cells in a three-dimensional arrangement to form a tissue cell-based structure, the tissue cell fluid having characteristics that allow the tissue cells to be deposited via the printer; and
a computing system coupled to the printer and configured to control the printer to deposit the tissue cells at selected positions defined by the arrangement.

2. The system of claim 1, wherein the printer is a piezoelectric inkjet printer including a sub-millimeter diameter nozzle that deposits the tissue cells at the selected positions in response to an electrical signal, and the computing system triggers the electrical signal to cause the nozzle to deposit the tissue cells at the selected positions.

3. (canceled)

4. The system of claim 1, wherein the tissue cell source provides a corneal collagen matrix with kerotocytes.

5. The system of claim 4, wherein the tissue cell-based structure formed by the arrangement is a corneal replacement, a corneal implant, or a spacer for corneal restructuring.

6. (canceled)

7. (canceled)

8. A system for producing a tissue-related structure, comprising:
a source including a photoreactive liquid precursor;
an application system coupled to the source and configured to deposit the photoreactive liquid precursor in one or more applications to form a three-dimensional polymer-based structure, the photoreactive liquid precursor having characteristics that allow the photoreactive liquid precursor to be deposited via the application system; and
an illumination system configured to deliver light to the photoreactive liquid precursor deposited by the application system and to solidify the photoreactive liquid precursor into the three-dimensional polymer-based structure.

9. The system of claim 8, further comprising a computing system coupled to the application system and the illumination system and configured to control the application system to deposit the photoreactive liquid precursor according to the one or more applications and to control the illumination system to deliver the light to the photoreactive liquid precursor deposited by the application system.

10. The system of claim 8, wherein the photoreactive liquid precursor includes a biocompatible photoinitiator to make the liquid precursor photoreactive.

11. The system of claim 10, wherein the biocompatible photoinitiator includes riboflavin and triethanolamine (TEOHA) and causes cross-linking activity with the photoreactive liquid precursor in response to the light from the illumination source.

12. The system of claim 10, wherein the photoreactive liquid precursor includes polyethylene glycol diacrylate, and the biocompatible photoinitiator causes cross-linking activity with the polyethylene glycol diacrylate in response to the light from the illumination source.

13. The system of claim 8, wherein the illumination system provides simultaneous absorption of more than one photon to deliver sufficient energy to solidify the photoreactive liquid precursor into the three-dimensional polymer-based structure.

14. The system of claim 8, wherein the polymer-based structure is a scaffold for seeding tissue cells for tissue cell growth.

15. The system of claim 14, wherein the scaffold is configured to allow the tissue cells to grow into a corneal replacement or a corneal implant.

16. (canceled)

17. The system of claim 8, wherein the polymer-based structure is a corneal implant.

18. The system of claim 8, wherein the polymer-based structure is a spacer for corneal restructuring.

19. The system of claim 8, wherein the polymer-based structure is a stent that is configured to relieve intraocular pressure for treating glaucoma.

20. The system of claim 8, wherein the application system includes a piezoelectric inkjet printer including a sub-millimeter diameter nozzle that deposits photoreactive liquid precursor at the selected positions in response to an electrical signal.

21. The system of claim 8, wherein the application system is configured to deposit the photoreactive liquid precursor in the eye, and the illumination device is configured to deliver the light to the photoreactive liquid precursor deposited in the eye.

22. The system of claim 21, wherein the illumination system provides simultaneous absorption of more than one photon to deliver sufficient energy to solidify the photoreactive liquid precursor into the three-dimensional polymer-based structure.

23. The system of claim 21, wherein the illumination device includes an optical fiber and a focusing lens to deliver the light to the photoreactive liquid precursor deposited in the eye.

24. The system of claim 21, wherein the illumination device includes a micro-manipulator that delivers the light according to a desired pattern to solidify the photoreactive liquid precursor into the three-dimensional polymer-based structure.

25-47. (canceled)
